(19) United States
(12) Patent Application Publication
(10) Pub. No.: US 2020/0030121 A1
(43) Pub. Date: Jan. 30, 2020

(54) ARTICULATED ORTHOPAEDIC FOOT WITH SHOCK ABSORPTION, WHICH PREVENTS THE IMPACT PRODUCED IN EACH FOOT-LOADING CYCLE WHEN WALKING OR RUNNING, PROVIDING NATURAL MOVEMENT AND STABILITY FOR THE USER

(71) Applicant: SOLUCIONES TAKEAHAND CHILE SPA, Santiago (CL)

(72) Inventors: Miguel Heli MORA MORALES, Santiago (CL); Oswaldo David DAVILA CARRASCO, Santiago (CL); Amweica SILVA FALCON, Santiago (CL)

(21) Appl. No.: 16/491,823

(22) PCT Filed: Mar. 5, 2018
(86) PCT No.: PCT/IB2018/051407

§ 371 Date: Sep. 6, 2019

(30) Foreign Application Priority Data
Mar. 6, 2017 (CL) 0535-2017

(51) Int. Cl. A61F 2/66

(52) U.S. Cl. CPC A61F 2/6607; A61F 2002/6642; A61F 2002/665; A61F 2002/5072; A61F 2002/6635; A61F 2002/6621; A61F 2002/6657

(57) ABSTRACT

The present invention is an articulated orthopaedic foot (100) with shock absorption, which prevents the impact produced in each foot-loading cycle when walking or running, providing natural movement and stability for the user, the foot being adaptable to irregular surfaces and comprising: a central pin (1) pivotably joined to a pair of metatarsal plates (2); an instep subsystem (101) solidly joined to the pair of metatarsal plates (2) by securing means; an ankle subsystem (102) pivotably joined to the central pin (1), metatarsal shock-absorbing means (40) being positioned between the ankle subsystem (102) and the instep subsystem (101); a calcaneus support (13) pivotably joined to the central pin (1), calcaneus shock-absorbing means (41) being positioned between the calcaneus support (13) and the ankle subsystem (102), to absorb impact when the user uses the articulated orthopaedic foot (100); and coupling means (20) positioned in the upper part of the ankle subsystem (102), for joining a tibia prosthesis to the articulated orthopaedic foot (100).

### FIELD OF THE INVENTION

The present invention relates to the orthopedic industry, in particular, the present invention relates to a prosthetic or orthopedic device. Such device is an articulated foot that allows a person with an amputated leg, including below and above the knee amputation, to walk naturally.

### BACKGROUND

Currently, in the prosthesis industry, there are many patents for prosthetic devices whose function is to perform the function of a human foot. The devices vary from simple shapes to complex elements with mechanical and robotic systems. Yet, the existing devices do not allow for the flexing, extension, and adaptability of a natural human foot. These prosthetic devices lack a number of aspects that can improve their function: both in the manner in which they are attached or adapted to a member, as in the way they function or dampen the impacts of typical actions such as walking, running, exercising, etc.

There have been several attempts to achieve a walking cycle that imitates that of a human foot, as evidenced in some patents, in relation to flexibility and to the joints that come into action whilst walking.

An example of such attempts can be found in U.S. Pat. No. 92,031 by James A. Foster, where a subdivision of the 3 parts of the foot (tarsus, metatarsus and phalanges) is already attempted, comprising a joint system for the movements in the walking cycle. This devise prioritizes the balance provided in the bodyweight support phase. Nevertheless, in such case the arrangement of the joint axis and of the damping element is inefficient, as it does not allow the foot to flex in the sole area, making the movement very rigid.

Similarly, U.S. Pat. No. 963,796 to E. Mueller discloses an orthopedic foot with a single pivot at ankle height that simulates the movement of the ankle and is dampened by a spring at the back and another at the front. Roche U.S. Pat. No. 2,430,584 also discloses an orthopedic foot with a single pivot at ankle height, which simulates the movement of the ankle and is dampened by a spring in the back and another in the front, additionally it includes a bending element in the metatarsal portion, to simulate the movement of the toes. Both patents try to imitate the balancing that occurs in the tarsus-metatarsal joint, but the position of the articular axis prevents the springs located in front and behind it to come into action; they do not properly soften the impact. Furthermore, given the position of the shaft at ankle height the received impact, typical of walking or running, is almost directly transferred to the user of the prosthesis.

The impact absorption in the heel purposed to try to smoothen the impact of the initial load in the walking cycle has not been implemented in the best way since they have located these cushions in very rigid sectors and are limited by the joint axes, which are the ones that actually receive the first impact of the load. This inefficiency can be found for example in the device for U.S. Pat. No. 6,666,895 to College Park Industries Inc, which comprises a pivot with a pair of shock absorbers that allow for foot mobility, yet it fails to solve the technical problem of softening the walking impact.

On the other hand, patent U.S. Pat. No. 5,913,902 discloses a foot prosthesis that has a pivot at ankle height with a pair of springs that allow the foot to move. The device also includes a spring in the metatarsus which severs the continuation of the movement generated by the inclination of the body of the user in its sagittal axis on the articulation Tarso-Metatarso. The aforementioned system produces a shearing force on the spring making the impact absorption inefficient. Additionally, the base of the foot has a restricted movement since it is embedded in a cavity, so it fails to solve the technical problem of the walking impact.

Biomechanically there is a gap in the existing prostheses, since each one attempts to solve punctual problems, sacrificing important aspects for the functioning of the foot as a whole, eventually causing a deterioration of the musculoskeletal system of amputees as previously mentioned.

Other articulated prostheses present problems with the use of shoes: either they include an elastomer covering which limits its efficiency and wear out rapidly, or they don't include it, and thus are aesthetically unpleasing to the user.

There is a need for a solution that covers all aspects not covered one-hundred-percent by existing prostheses, and that can adapt to shoe sizes and can also be used barefoot.

Therefore, there is a need to have an orthopedic foot that avoids the damages caused by the impact that occurs in each weight-load cycle of the foot. It is one of the problems that adds to an unnatural walk in the field of prosthetic or orthopedic feet. This impact directly affects the rest of the skeletal muscle structure and also generates irregularity in the natural way of walking.

### Solution to the Outlined Technical Problem

To remedy the problem, we present an articulated orthopedic foot that prevents the impact that occurs in each load cycle of the foot when walking or running, reducing the impact and deterioration of the rest of the bone structure of the user with leg or foot absence, additionally providing a more natural foot-movement and stability, whether walking, running, or just standing. This orthopedic foot is adaptable to irregular surfaces and can be worn with or without a shoe, without requiring adapters.

### DESCRIPTION OF THE FIGURES

FIG. 1 shows a side view of a preferred configuration of an articulated orthopedic foot (100) with an instep subsystem (101) and an ankle subsystem (102) with an articulated calcaneal support (13).

FIG. 2 shows a side view of another preferred configuration of the articulated orthopedic foot (100) with the instep subsystem (101) with a joint and the ankle subsystem (102) with the articulated calcaneal support (13).

FIG. 3 shows a side view of another preferred configuration of the articulated orthopedic foot (100) with the instep subsystem (101) and the ankle subsystem (102) with a hinge and with the hinged calcaneus (13).

FIG. 4 shows a side view of another preferred configuration of the articulated orthopedic foot (100) with the subsystem subsystem (101) with the joint and the ankle subsystem (102) with another joint and with the articulated calcaneal support (13).

FIG. 5 shows a side view of a central pin (1) pivotally attaching metatarsal plates (2) and fixed ankle plates (3)

FIG. 6 shows a side view of the articulated orthopedic foot (100) with coupling means (20), coupling pins (20′), spaces for metatarsal damping elements (30), spaces for calcaneus damping elements (31) and spaces for phalange damping elements (32).

FIG. 7 shows a top view of the metatarsal plates (2) and the fixed ankle plates (3).

FIG. 8 shows a side view of another configuration of the central pin (1) that pivotally attaches metatarsal plates (2), the fixed ankle plates (3) with supporting plates (4).

FIG. 9 shows a top view of the articulated orthopedic foot (100).

FIG. 10 shows a side view of the articulated orthopedic foot (100) with a metatarsal damping elements (40) and a calcaneus damping elements (41).

FIG. 11 shows a rear view of the articulated orthopedic foot (100) and the calcaneus damping elements (41).

FIG. 12 shows a cross-sectional view of the articulated orthopedic foot (100) and rear calcaneal angles (D, D′).

FIG. 13 shows a cross-sectional view of the articulated orthopedic foot (100) and lateral calcaneal angles (C, C′), phalange angles (E) and non-slip means (25).

FIG. 14 shows a side view of the articulated orthopedic foot (100) with a metatarsal cap (21) and a calcaneal cap (22).

FIG. 15 shows a side view of the articulated orthopedic foot 100 in another configuration with the metatarsal cap (21), calcaneal cap (22), ankle cap (23) and phalange cap (24).

FIG. 16 shows a rear view of the articulated orthopedic foot (100) with the calcaneal cap (22).

FIG. 17 shows a top view of the articulated orthopedic foot (100) with the metatarsal cap (21), the calcaneal cap (22), an ankle cap (23) and a phalangeal cap (24).

FIG. 18 shows an isometric view of the articulated orthopedic foot 100 with the metatarsal cap (21), the calcaneal cap (22), an ankle cap (23) and a phalange cap (24).

FIG. 19 shows a side view of another preferred configuration of the metatarsal plates (2), the lower mobile ankle plates (6), the upper mobile ankle plates (7) and a phalangeal pin (8) for raising the mechanical strength.

FIG. 20 shows a top view of the metatarsal plates (2), the lower mobile ankle plates (6) and the upper mobile ankle plates (7).

FIG. 21 shows a side view of another preferred configuration of the metatarsal plates (2), lower mobile ankle plates (6) and upper mobile ankle plates (7).

FIG. 22 shows a cross-sectional view of the articulated orthopedic foot (100), with the detail of a metatarsal angle (A), an ankle angle (B), a lateral calcaneus angle (C, C′) and a phalange angle (E).

FIG. 23 shows a cross-sectional view of the articulated orthopedic foot (100), with the detail of a rear calcaneus angle (D, D′).

FIG. 24 shows a side view of the articulated orthopedic foot (100), spaces for metatarsal damping elements (30) and a space for ankle damping elements (33).

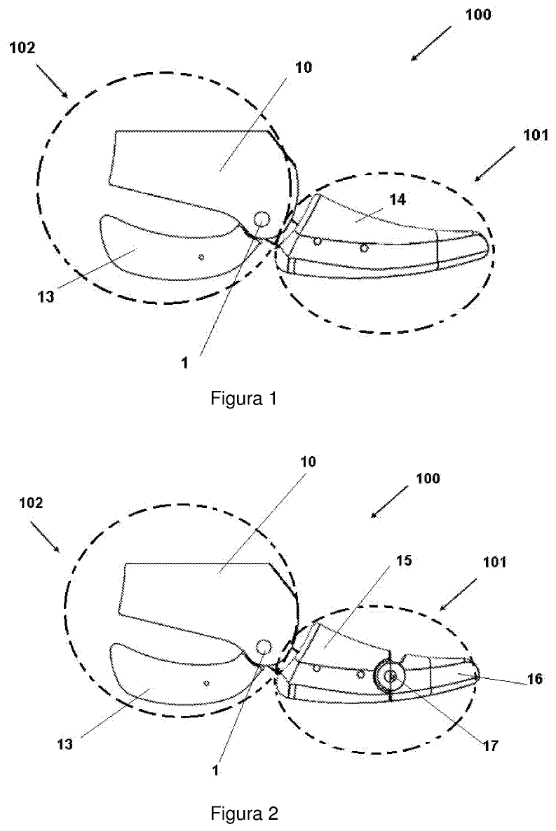

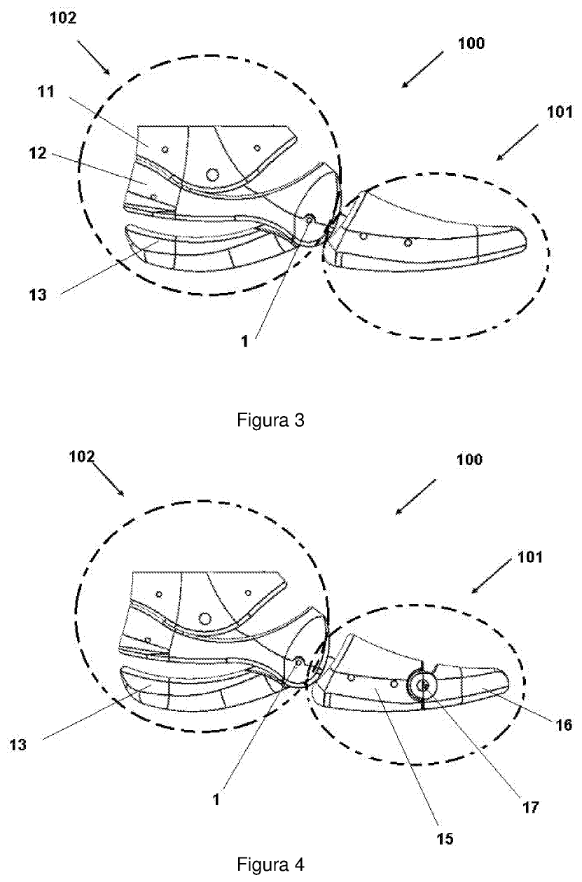

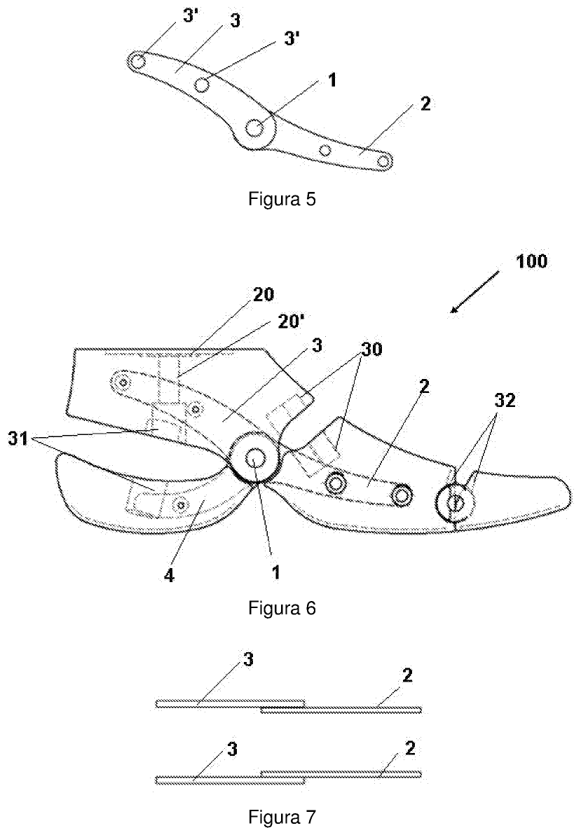

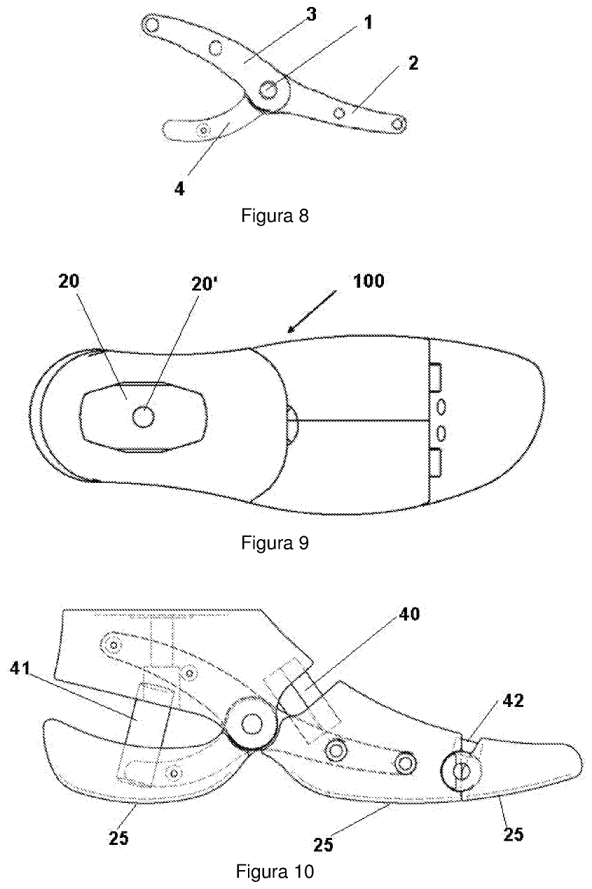

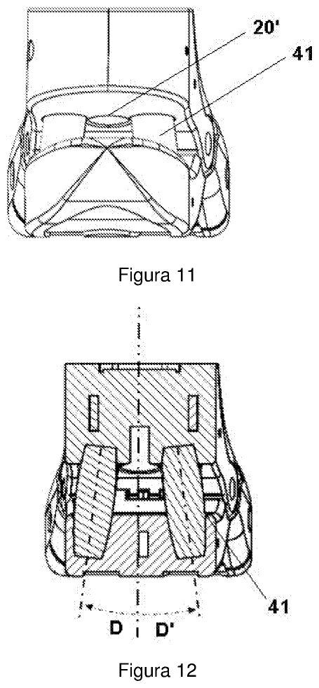

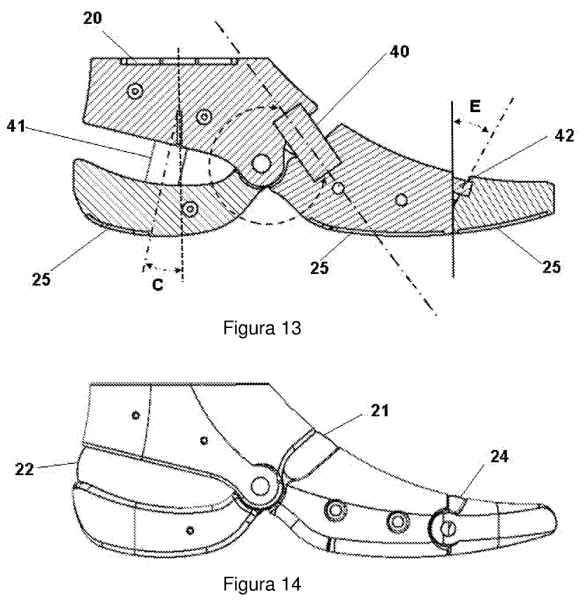

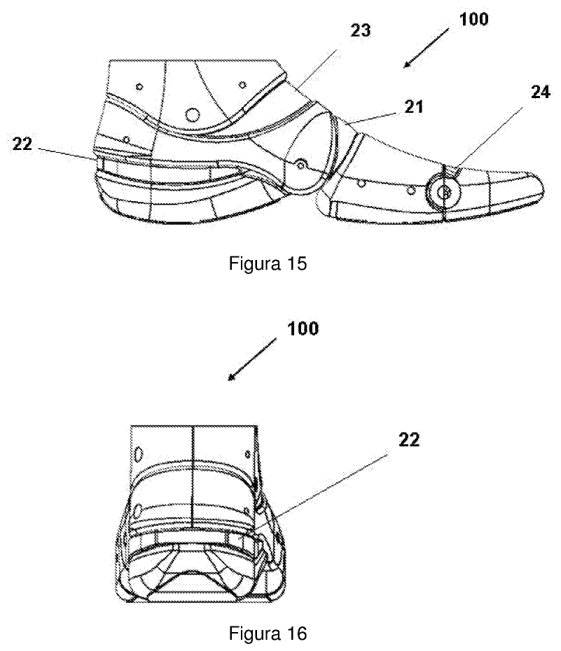

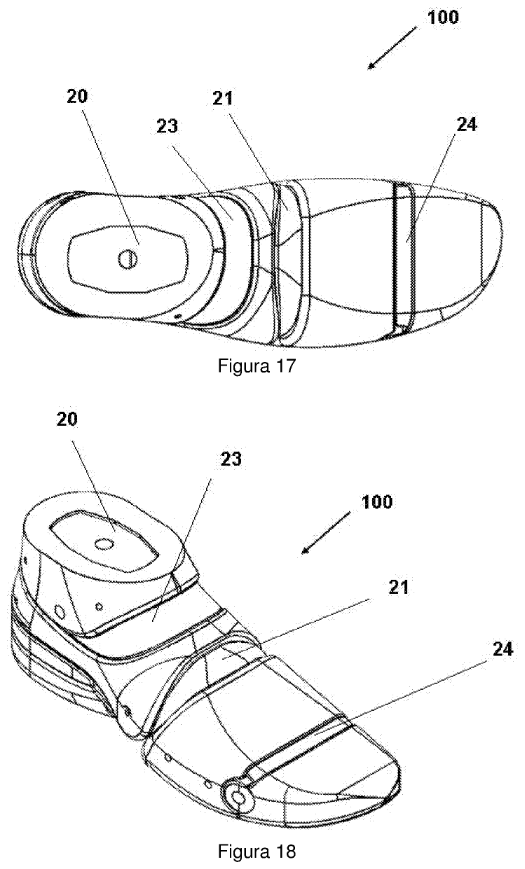

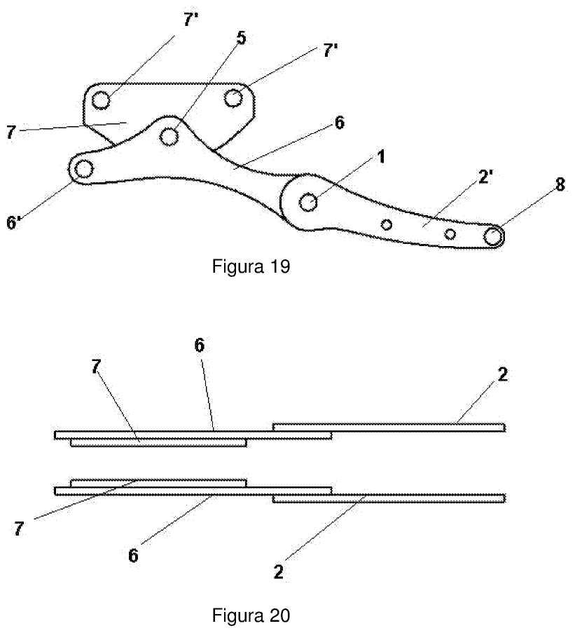

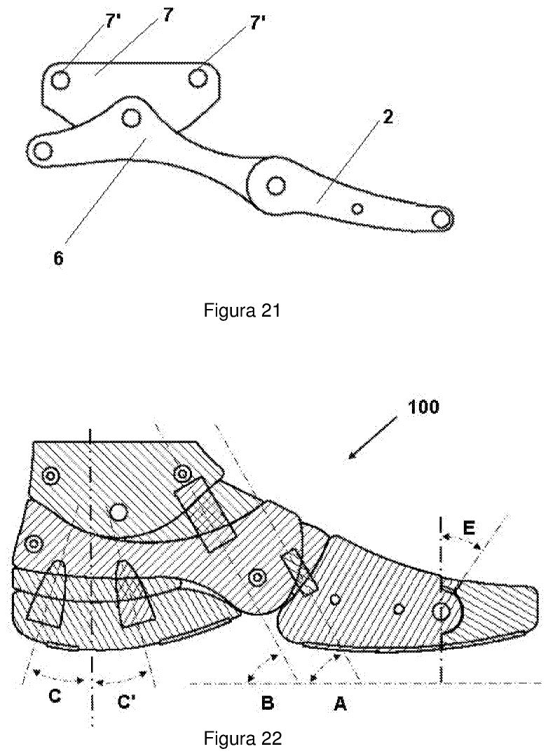

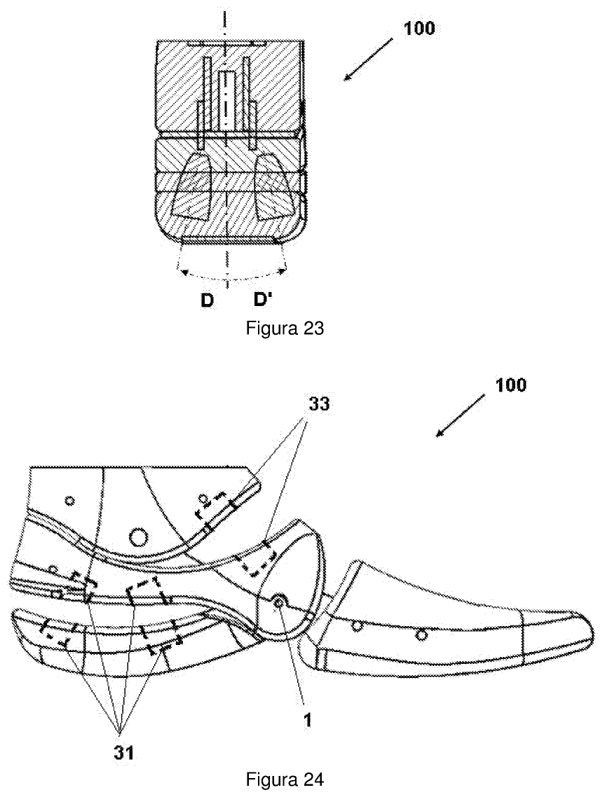

### DESCRIPTION OF THE INVENTION

The device we are disclosing is an articulated orthopedic foot (100) which minimizes the impact that occurs in each foot loading cycle when walking or running, which comprises a set of joints uniting the parts of the articulated orthopedic foot, which allow a better distribution of loads and flexing movements of the foot, better adapting to the pace of the march.

The set of dampers that allow the flexion and dynamic self-extension of the foot is strategically located with angular inclinations that allow to better receive and distribute the weight loads. The weight loads at the moment of the walk are made cyclically and follow the orbit of the joint axis that links Tarso-Metatarso-Heel, taking this joint as a pivot, which is in front of the ankle axis to balance the body forward in the support phase whilst the step is being taken with the other foot.

Between the heel and the metatarsus, a dynamic arch is created that opens at the moment of the plantar support and closes when the foot is lifted, providing increased softening since it simulates the arch of the foot.

The damping element, placed on the heel, has a double inclination: the first in relation to the front axle, which allows to receive the initial weight load of the walking cycle in the best position and to preload to give the impulse to raise the foot after the body rolls during the plantar support phase. The second inclination is between damping elements, being side by side on the front axle, they allow the weight-load to be distributed and to generate balancing on uneven surfaces, which mimics the flexibility provided by the human ankle to adapt to inclinations and irregularities.

The damping elements placed between the Tarsus and Metatarsus allows to flex and extend in the balancing during the plantar support phase thanks to the degrees of longitudinal inclination in respect to the instep of the foot and the axis of the joint. Additionally, in the case of transtibial amputation, the inclination that is generated in the rest of the prosthesis facilitates the flexing of the knee, avoiding wrongdoings in the walking cycle, as it helps to activate all the joints and muscles involved in walking.

Similarly, the metatarsal-phalanges joint has a system to recover the original position with impact absorption after the deployment of the foot in the last phase of support. The phalange has an angle cut in the direction of the frontal axis, which allows for the necessary mobility to finish the march.

Among the extra features to be pointed out in this invention is the adaptability to the structural system of traditional prostheses and the pyramidal alignment systems.

The articulated orthopedic foot (100) presented here can be scaled to any desired size, for example, from size 20 to size 50, to smoothly fit into different types of shoes.

A safety cap that gives formal continuity to the foot, covers the spaces and masks each damping system. These are made with a flexible material that does not interfere in the functioning of the joints.

Non-slip and flexible material is placed at the surface of the foot base that ensure a more secure and smooth step.

### DETAILED DESCRIPTION OF THE INVENTION

As shown in the figures, the present invention discloses an articulated orthopedic foot (100) with a damping element, which avoids the shock impact that occurs in each cycle of weight loading on the foot when walking or running and is adaptable to irregular surfaces, delivering a natural movement and stability. The device comprises:

a central pin (1), which is pivotally attached to a pair of metatarsal plates (2);

an instep subsystem (101) which is integrally attached to the pair of metatarsal plates (2), through fastening means;

an ankle subsystem (102) pivotally attached to the central pin (1), where means for metatarsal damping (40) are located between the ankle subsystem (102) and the instep subsystem (101);

a calcaneal support (13) pivotally attached to the central pin (1), where means for calcaneal damping (41) are located between the calcaneal support (13) and the ankle subsystem (102), to absorb the impact when the user uses this articulated orthopedic foot (100); and coupling means (20) are found at the top of the ankle subsystem (102) to attache a tibial prosthesis to the articulated orthopedic foot (100).

Furthermore, it comprises a pair of support plates (4) which are attached pivotally to the central pin (1) and integrally to the calcaneal support (13) through fastening means, to increase the mechanical resistance of the articulated orthopedic foot (100).

In a preferred configuration the ankle subsystem (102) is fixed, forming a fixed ankle (10).

To increase the mechanical strength of the articulated orthopedic foot (100), a pair of fixed ankle plates (3) are included, which are pivotally attached to the central pin (1) and integrally secured to the fixed ankle (10).

In a preferred configuration, the instep subsystem (101) is fixed, forming a fixed metatarsal (14), which is integrally attached to the metatarsal plates (2), and in another preferred configuration the instep subsystem (101) is mobile, comprising:

a mobile metatarsus (15), which is integrally attached to the metatarsal plates (2);

a phalange (16), which is pivotally attached to the mobile metatarsus (15), by means of a phalangeal pin (17);

a phalange damping mean (42) located between the mobile metatarsus (15) and the phalange (16), to absorb the impact and make the movement more natural at the time of using this articulated orthopedic foot (100), wherein the metatarsal plates (2) additionally comprise a metatarsal platen pin (2′) with a phalangeal pin (8), through which the phalangeal pin (17) passes, to give greater structural strength to the instep subsystem (101).

In another preferred configuration, the ankle subsystem (102) is mobile, which comprises:

At least two mobile lower ankle plates (6), which are pivotally attached to the central pin (1), wherein each of the mobile lower ankle plate (6) joins an mobile upper ankle plate (7) in a pivoting manner by means of a pin (5);

a lower mobile ankle (12) is integrally attached to the lower mobile ankle plates (6) through fastening means; and

a mobile upper ankle (11) is integrally attached to the upper mobile ankle plates (7); ankle damping system (43) are located between the upper mobile ankle (11) and the lower mobile ankle (12) to absorb the impact and provide a more natural foot-movement when the user uses this articulated orthopedic foot (100).

The articulated orthopedic foot (100) achieves its greatest flexibility when the instep subsystem (101) is mobile, which comprises:
a mobile metatarsus (15), which is integrally attached to the metatarsal plates (2); a phalange (16) which is pivotally attached to the mobile metatarsus (15), by means of a phalangeal pin (17);

The phalange damping elements (42) located between the mobile metatarsal (15) and the phalange (16) to absorb the impact and provide a more natural foot-movement when the user uses this articulated orthopedic foot (100); and the ankle subsystem (102) is mobile, which comprises:

at least two lower mobile ankle plates (6), which are pivotally attached to the central pin (1), wherein each of the lower mobile ankle plates (6) pivotally joins an upper mobile ankle plate (7) by means of a pin (5);

a lower mobile ankle (12) is integrally attached to the lower mobile ankle plates (6) through fastening means; and

an upper mobile ankle (11) is integrally attached to the upper mobile ankle plates (7); ankle damping elements (43) are located between the upper mobile ankle (11) and the lower mobile ankle (12) to absorb the impact and provide a more natural foot-movement when the user uses this articulated orthopedic foot (100).

a lower mobile ankle (12) is integrally attached to the lower mobile ankle plates (6) through fastening means; and

an upper mobile ankle (11) is integrally attached to the upper mobile ankle plates (7); ankle damping elements (43) are located between the upper mobile ankle (11) and the lower mobile ankle (12) to absorb the impact and provide a more natural foot-movement of the user at the time of using this articulated orthopedic foot (100).

In another configuration, the metatarsal plates (2) additionally comprise a metatarsal plate pin (2′) with a phalangeal pin (8), through which the phalangeal pin (17) passes, to give greater structural strength to the instep subsystem (101).

For safety reasons, a metatarsal cap (21) is located above the metatarsal damping elements (40) and a calcaneal cap (22) is located above the calcaneus damping elements (41) to avoid pinching accidents between the moving parts related to said metatarsal damping elements (40) and the calcaneus damping elements (41).

In addition, an ankle cap (23) is positioned above the ankle dampening elements (43) to prevent pinch accidents between the moving parts related to said ankle dampening elements (43), and a phalange cap (24) is located above the phalange damping elements (42) to prevent pinching accidents between the moving parts related to said phalange damping elements (42).

In order to increase grip when walking, running, playing, jumping, etc., when using the present articulated orthopedic foot (100) barefoot, anti-slip means (25) are placed below the calcaneal support (13) and below the instep subsystem (101), wherein the anti-slip means (25) are composed of rubber, sole, suction cups, cleats, polyurethane plants, snow or ice tips or any other anti-slip means, to improve adhesion to the floor.

In another preferred configuration, the metatarsal damping elements (40) have a metatarsal angle (A) with respect to the horizontal between 30° and 90° and more preferably the metatarsal angle (A) with respect to the horizontal is 60°±15°.

In another preferred configuration, the calcaneus damping elements (41) have a lateral (C, C′) calcane angle with respect to the vertical between −10° and 30°, and more preferably the lateral calcaneus angle (C, C′) with respect to the vertical is of 10°±15°, where the lateral calcaneal angle (C) may be equal to or different from the lateral calcaneal angle (C′).

In another preferred configuration, the calcaneus damping elements (41) have a rear calcaneus angle (D, D′) with respect to the vertical between −10° and 30°, and more preferably the rear calcaneus angle (D, D′) with respect to the vertical is of 10°±15°, where the rear calcaneal angle (D) may be equal to or different from the posterior calcaneal angle (D′).

In another preferred configuration, the ankle dampening elements (43) have an ankle angle (B) with respect to the horizontal between 30° and 90°, and more preferably the ankle angle (B) with respect to the horizontal is 60°±15°.

In another preferred configuration, the phalange damping elements (42) have an angle (E) with respect to the vertical between 0° and 60°, and more preferably the angle (E) with respect to the vertical is 30°±10°.

The metatarsal damping elements (40) joins in a flexible manner the instep subsystem (101) with the ankle subsystem (102); the phalange damping elements (42) joins in a flexible manner the mobile metatarsus (15) to the phalange (16); the ankle damping elements (43) joins in a flexible manner to the upper mobile ankle (11) and the lower mobile ankle (12), preventing them from separating.

The metatarsal damping elements (40), the calcaneus damping elements (41), the phalange damping elements (42) and the ankle damping elements (43) are made of a flexible material which is selected from: elastomer, viscoelastic or a type of flexible plastic, polyurethane, gum or rubber, or can also be compression springs, or mixtures between the different materials and damping elements.

The metatarsus damping elements (40), the calcaneus damping elements (41), the phalange damping elements (42) and the ankle damping elements (43) are in the form of cylinders or truncated or convex cones, which are placed in respective channels to prevent them from coming out, these can be united by a cotter pin, glue or by wedge.

In another preferred configuration, the metatarsal damping elements (40) and/or the phalange damping elements (42) and/or the ankle damping elements (43) are tensile springs (not shown in the figures).

In another preferred configuration the metatarsal damping elements (40) and/or the phalange damping elements (42) and/or the ankle damping elements (43) are constructed of flexible tensile materials (not shown in the figures).

The coupling means (20) may be quick couplings, bolts, screws, glue, hooks, mechanical locks or any other mechanical fastening means, and in yet another preferred configuration, these coupling means (20) comprise (20′), so as to passably insert the bolts, screws, among other types of similar fastening means.

On the other hand, the fixed ankle plates (3) additionally comprise at least one perforation of fixed ankle plates (3′), the lower mobile ankle plates (6) additionally comprise at least one perforation of lower mobile ankle plates (6′) and the upper mobile ankle plates (7) additionally comprise at least one upper mobile ankle plate (7′) perforation, wherein all of these perforations are to place through them pins, bolts, screws or other fixating means for increasing the mechanical strength of the articulated orthopedic foot (100).

### Example of Application

The present articulated orthopedic foot (100) with impact absorption was used by a 35-year-old 85-kg person who, when wearing the present orthopedic device, was able to walk and run, first without a sneaker and then with a sneaker. In addition, this person is a gym teacher, who used it to jump and support the foot on top of a flexible plastic ball of approximately 80 cm diameter. The exercise consists of supporting one foot on top of the ball and the other on the floor alternating, and in each change of foot a jump must be performed. The user was able to perform this exercise without problems and the articulated orthopedic foot (100) supported the mechanical weight loading of this exercise without inconveniences.

It is therefore concluded that the present articulated orthopedic foot is easy to use and accommodates the needs of users, providing impact absorption and a natural foot movement.

List of parts: The present list is to facilitate the understanding of the device to the reader.
(A) Metatarsal angle (A)
(B) Ankle angle (B)
(C, C′) Lateral calcaneus angle (C, C′)
(D, D′) Rear calcaneus angle (D, D′)
(E) Phalange angle (E)
(1) Center pin (1)
(2) Metatarsal plates (2);
(2′) Metatarsal plate pin (2′)
(3) Fixed ankle plates (3)
(3′) Perforation of fixed ankle plates (3′)
(4) Supporting plates (4)
(5) Pin (5)
(6) Lower mobile ankle plates (6)
(6′) Perforation of lower mobile ankle plates (6′)
(7) Upper mobile ankle plate (7)
(7′) Perforation of upper mobile ankle plate (7′)
(8) Phalange pin (8)
(10) Fixed ankle (10)
(11) Upper mobile ankle (11)
(12) Lower mobile ankle (12).
(13) Calcaneal Support (13)
(14) Fixed metatarsal (14)
(15) Mobile metatarsal (15)
(16) Phalange (16)
(17) Phalangeal pin (17);
(20) Coupling means (20)
(20′) Coupling pins (20′)
(21) Metatarsal cap (21)
(22) Calcaneal cap (22)
(23) Ankle cap (23)
(24) Phalange cap (24)
(25) Anti-slip means (25)
(30) Space for metatarsal damping elements (30)
(31) Space for calcaneus damping elements (31)
(32) Space for phalange damping elements (32)
(33) Space for ankle damping elements (33)
(40) Metatarsals damping elements (40)
(41) Calcium damping elements (41)
(42) Phantom damping elements (42)
(43) Ankle damping elements (43)
(100) Articulated orthopedic foot (100)
(101) Instep subsystem (101)
(102) Ankle subsystem (102)

1. An articulated orthopedic foot (100) with impact absorption, which minimizes the impact that occurs in each weight load cycle of the foot when walking or running, delivering a natural movement and stability for a user, which is adaptable to irregular surfaces, CHARACTERIZED by comprising:
a central pin (1), which is pivotally attached to a pair of metatarsal plates (2);
an instep subsystem (101), which is integrally attached to the pair of metatarsal plates (2) through fastening means;
an ankle subsystem (102), which is pivotally attached to the central pin (1), wherein a metatarsal damping elements (40) is located between the ankle subsystem (102) and the instep subsystem (101);
a calcaneal support (13), which is pivotally attached to the central pin (1), wherein calcaneal damping elements (41) are located between the calcaneal support (13) and the ankle subsystem (102), to absorb the impact when the user uses this articulated orthopedic foot (100); and
a coupling mean (20) located at the top of the ankle subsystem (102) for attaching a tibial prosthesis to the articulated orthopedic foot (100).

2. The orthopedic orthopedic foot (100) with impact absorption according to claim 2, CHARACTERIZED by additionally comprising a pair of support plates (4) which are pivotally attached to the central pin (1) and integrally with the calcaneus support (13) through fastening means, to increase the mechanical resistance of the articulated orthopedic foot (100).

3. The articulated orthopedic foot (100) with impact absorption, according to claim 1, CHARACTERIZED by the ankle subsystem (102) being fixed, forming a fixed ankle (10).

4. The cushioned articulated orthopedic foot (100) according to claim 2, CHARACTERIZED by additionally comprising a pair of fixed ankle plates (3), which are pivotally attached to the central pin (1) and integrally with the ankle (10) to increase the mechanical strength of the articulated orthopedic foot (100).

5. The articulated orthopedic foot (100) with impact absorption, according to claim 1, CHARACTERIZED by the instep subsystem (101) being fixed, forming a fixed metatarsal (14), which is integrally attached to the metatarsal plates (2).

6. The cushioned articulated orthopedic foot (100) according to claim 1, CHARACTERIZED by the instep subsystem (101) being mobile, which comprises:
a mobile metatarsus (15), which is integrally attached to the metatarsal plates (2);
a phalange (16), which is pivotally attached to the mobile metatarsus (15), by means of a phalangeal pin (17);
phalange damping elements (42) which are located between the mobile metatarsal (15) and the phalange 1(6) to absorb the impact and provide a more natural foot-movement when the user uses this articulated orthopedic foot (100).

7. The articulated orthopedic foot (100) with impact absorption according to claim 6, CHARACTERIZED by the metatarsal plates (2) additionally comprising a metatarsal plate pin (2′) with a phalangeal pin (8), by which passes the phalangeal pin (17), to give greater structural strength to the instep subsystem (101).

8. The cushioned articulated orthopedic foot (100) according to claim 1, CHARACTERIZED by the ankle subsystem (102) being mobile, which comprises:
at least two lower mobile ankle plates (6), which are pivotally attached to the central pin (1), wherein each of the lower mobile ankle plate (6) joins an upper mobile ankle plate (7) in a pivoting manner by means of a pin (5);
a lower mobile ankle (12) is integrally attached to the lower mobile ankle plates (6) through fastening means; and
an upper mobile ankle (11) is integrally attached to the upper mobile ankle plates (7); and ankle damping elements (43) are located between the upper mobile ankle (11) and the lower mobile ankle (12) to absorb the impact and provide a more natural foot-movement when the user uses this articulated orthopedic foot (100).

9. The cushioned articulated orthopedic foot (100) according to claim 1, CHARACTERIZED by the instep subsystem (101) being mobile, which comprises:
a mobile metatarsus (15), which is integrally attached to the metatarsal plates (2);
a phalange (16), which is pivotally attached to the mobile metatarsus (15), by means of a phalangeal pin (17);
phalange damping elements (42), which are located between the mobile metatarsal (15) and the phalange (16) to absorb the impact and provide a more natural foot-movement when the user uses this articulated orthopedic foot (100); and
the ankle subsystem (102) is mobile, which comprises:
at least two lower mobile ankle plates (6), which are pivotally attached to the central pin (1), wherein each of the lower mobile ankle plate (6) joins an upper mobile ankle plate (7) in a pivoting manner by means of a pin (5);
a lower mobile ankle (12) is integrally attached to the lower mobile ankle plates (6) through fastening means; and
an upper mobile ankle (11) is integrally attached to the upper mobile ankle plates (7); and ankle damping elements (43) are located between the upper mobile ankle (11) and the lower mobile ankle (12) to absorb the impact and provide a more natural foot-movement when the user uses this articulated orthopedic foot 100.

10. The articulated orthopedic foot (100) with impact absorption according to claim 9, CHARACTERIZED by the metatarsal plates (2) additionally comprising a metatarsal plate pin (2′) with a phalangeal pin (8), by which passes the phalangeal pin (17), to give greater structural strength to the instep subsystem (101).

11. The articulated orthopedic foot (100) with impact absorption, according to claim 1, CHARACTERIZED by a metatarsal cap (21) being located above the metatarsal damping elements (40) and a calcaneal cap (22) being located above the calcaneus damping elements (41) to prevent pinching accidents between the moving parts related to said metatarsal damping elements (40) and the calcaneus damping elements (41).

12. The orthopedic orthopedic foot (100) with impact absorption according to claim 8 or 9, CHARACTERIZED by an ankle cover (23) being located above the ankle dampening elements (43), to avoid pinching accidents between the parts mobile relative to said ankle dampening elements (43).

13. The orthopedic orthopedic foot (100) with impact absorption, according to claim 6 or 9, CHARACTERIZED by a phalange cap (24) being located above the phalange damping elements (42), in order to avoid accidents by tightening between the parts mobile relative to said phalange damping elements (42).

14. The orthopedic orthopedic foot (100) with impact absorption according to claim 1, 5, 6, or 9, CHARACTERIZED by comprising non-slip means (25), located below the calcaneal support (13) and below the subsystem subsystem (101).

15. The articulated orthopedic foot (100) with impact absorption, according to claim 1, CHARACTERIZED by the metatarsus damping elements (40) having a metatarsal angle (A) with respect to the horizontal between 30° and 90°.

16. The articulated orthopedic foot (100) with impact absorption, according to claim 1, CHARACTERIZED by the metatarsus damping elements (40) having a metatarsal angle (A) with respect to the horizontal is 60°±15°.

17. The articulated orthopedic foot (100) with damping according to claim 1, CHARACTERIZED by the calcaneus damping elements (41) having a lateral calcaneus angle (C, C′) with respect to the vertical between −10° and 30°.

18. The articulated orthopedic foot (100) with damping according to claim 1, CHARACTERIZED by the calcaneus damping elements (41) having a lateral calcaneus angle (C, C′) with respect to the vertical of 10°±15°.

19. The cushioned orthopedic foot (100), according to claim 1, is CHARACTERIZED by the calcaneus damping elements (41) having a rear calcaneus angle (D, D′) with respect to the vertical between −10° and 30°.

20. The articulated orthopedic foot (100) with damping according to claim 1, CHARACTERIZED by the calcaneus damping elements (41) having a rear calcaneus angle (D, D′) with respect to the vertical of 10°±15°.

21. The orthopedic orthopedic foot (100) with impact absorption according to claim 8 or 9, CHARACTERIZED by the ankle damping elements (43) having an ankle angle (B) with respect to the horizontal between 30° and 90°.

22. The orthopedic orthopedic foot (100) with damping according to claim 8 or 9, CHARACTERIZED by the ankle damping elements (43) having an ankle angle (B) with respect to the horizontal of 60°±15°.

23. The orthopedic orthopedic foot (100) with impact absorption according to claim 8 or 9, CHARACTERIZED by the phalange damping elements (42) having an angle (E) with respect to the vertical between 0° and 60°.

24. The articulated orthopedic foot (100) with impact absorption according to claim 8 or 9, CHARACTERIZED by the phalange damping elements (42) having an angle (E) to the vertical of 30°±10°.

25. The cushioned articulated orthopedic foot (100) according to claim 1, CHARACTERIZED by the metatarsus damping elements (40) joining in a flexible manner the instep subsystem (101) with the ankle subsystem (102), preventing separation between them.

26. The orthopedic orthopedic foot (100) with impact absorption according to claim 6, CHARACTERIZED by the phalange damping elements (42) joining in a flexible manner the mobile metatarsus (15) with the phalange (16), avoiding the separation between them.

27. The orthopedic orthopedic foot (100) with impact absorption according to claim 8, CHARACTERIZED by the ankle damping elements (43) flexibly joining the upper mobile ankle (11) and the mobile ankle base (12), avoiding separation between them.

28. The orthopedic orthopedic foot (100) with impact absorption, according to claim 1, CHARACTERIZED by the metatarsus damping elements (40), the calcaneus damping elements (41), the phalange damping elements (42) ankle damping elements (43) being made of a flexible material.

29. The articulated orthopedic foot (100) with impact absorption, according to claim 25, CHARACTERIZED by the flexible material being elastomeric.

30. The articulated orthopedic foot (100) with impact absorption, according to claim 25, CHARACTERIZED by the flexible material being viscoelastic.

31. The articulated orthopedic foot (100) with impact absorption, according to claim 25, CHARACTERIZED by the flexible material being a type of flexible plastic, polyurethane, gum or rubber.

32. The articulated orthopedic foot (100) with impact absorption according to claims 1, 6 and 8, CHARACTERIZED by the metatarsus damping elements (40), the calcaneus damping elements (41), the phalange damping elements (42) and the ankle damping elements (43) being compression springs.

33. The articulated orthopedic foot (100) with impact absorption according to claims 1, 6 and 8, CHARACTERIZED by the metatarsus damping elements (40), the calcaneus damping elements (41), the phalange damping elements (42) and the ankle damping elements (43) being in the form of cylinders or truncated or convex cones which are located in respective channels so that these damping elements do not come out.

34. The articulated orthopedic foot (100) with impact absorption according to claims 1, 6 and 8, CHARACTERIZED by the metatarsus damping elements (40), the calcaneus damping elements (41), the phalange damping elements (42) and the ankle damping elements (43) being in the form of cylinders or truncated or convex cones.

35. The articulated orthopedic foot (100) with impact absorption, according to claim 1, CHARACTERIZED by the metatarsus damping elements (40) being tensile springs.

36. The articulated orthopedic foot (100) with impact absorption, according to claim 6, CHARACTERIZED by the phalange damping elements (42) being tensile springs.

37. The articulated orthopedic foot (100) with impact absorption, according to claim 8, CHARACTERIZED by the ankle damping elements (43) being tensile springs.

38. The orthopedic orthopedic foot (100) with impact absorption, according to claim 1, CHARACTERIZED by the coupling means (20) being quick couplings, bolts, screws, glue, hooks or mechanical locks.

39. The orthopedic orthopedic foot (100) with impact absorption, according to claim 1, CHARACTERIZED by the engaging means (20) comprising dowel pins (20′) for locating the pins or screws through them.

40. The orthopedic orthopedic foot (100) with impact absorption according to claim 2, CHARACTERIZED by the fixed ankle plates (3) additionally comprising at least one bore of fixed ankle plates (3′), which are purposed to insert bolts or screws, to increase the mechanical strength of the articulated orthopedic foot (100).

41. The orthopedic orthopedic foot (100) with impact absorption according to claim 8 or 9, CHARACTERIZED by the lower mobile ankle plates (6) additionally comprising at least one perforation of lower mobile ankle plates (6′), which are purposed to insert the pins, bolts or screws, to increase the mechanical strength of the articulated orthopedic foot (100).

42. The orthopedic orthopedic foot (100) with impact absorption according to claim 8 or 9, CHARACTERIZED by the upper mobile ankle plates (7) additionally comprising at least one upper mobile ankle plate perforation (7′), which are purposed to insert pins, bolts or screws, to increase the mechanical strength of the articulated orthopedic foot (100).

43. The cushioned articulated orthopedic foot (100) according to claim 14, CHARACTERIZED by the non-slip means (25) being of rubber, sole, suction cups, cleats, polyurethane plants, snow tips or ice to improve adhesion to floor.

44. The articulated orthopedic foot (100) with impact absorption, according to claim 17, CHARACTERIZED by the lateral calcaneal angle (C) being different from the lateral calcaneal angle (C′).

45. The articulated orthopedic foot (100) with impact absorption, according to claim 17, CHARACTERIZED by the lateral calcaneal angle (C) being equal to the lateral calcaneal angle (C′).

46. The articulated orthopedic foot (100) with impact absorption, according to claim 19, CHARACTERIZED by the rear calcaneal angle (D) being different from the rear calcaneal angle (D′).

47. The articulated orthopedic foot (100) with impact absorption, according to claim 19, CHARACTERIZED by the rear calcaneal angle (D) being equal to the rear calcaneal angle (D′).
